(19) United States
(12) Patent Application Publication
(10) Pub. No.: US 2019/0015346 A1
(43) Pub. Date: Jan. 17, 2019

(54) SELF-EMULSIFYING DRUG DELIVERY SYSTEM

(71) Applicant: PHARMACANNIS LABS LLC, Oak Park, IL (US)

(72) Inventor: Christopher Diorio, Campbell Hall, NY (US)

(73) Assignee: PHARMACANNIS LABS LLC, OAK PARK, IL (US)

(21) Appl. No.: 16/015,907

(22) Filed: Jun. 22, 2018

Related U.S. Application Data
(60) Provisional application No. 62/532,606, filed on Jul. 14, 2017.

(51) Int. Cl. A61K 9/48; A61K 31/352; A61K 31/05; A61P 29/02

(52) U.S. Cl. CPC A61K 9/4858; A61K 9/4808; A61P 29/02; A61K 31/05; A61K 31/352

(57) ABSTRACT

Formulations for oral administration of water-insoluble cannabinoids are disclosed. More particularly, self-emulsifying drug delivery systems for oral administration of water-insoluble, lipophilic cannabinoids are disclosed.

### RELATED APPLICATIONS

This application claims priority to U.S. Provisional Application Ser. No. 62/532,606 filed on Jul. 14, 2017, which is hereby incorporated by reference in its entirety.

### FIELD OF THE INVENTION

The embodiments of the present invention relate to the delivery of cannabinoids via a self-emulsifying drug delivery system.

### BACKGROUND OF THE INVENTION

Public interest in the medicinal use of cannabis has grown exponentially over the past decade. Cannabis sativa is an annual plant belonging to the Cannabaceae family. It contains more than 400 chemicals and approximately 80 cannabinoids, the active constituents of cannabis, including tetrahydrocannabinol (THC), cannabidiol (CBD), cannabinol (CBN), tetrahydrocannabivarin (THCV) and cannabigerol (CBG). Pharmacologically, the principal psychoactive constituent of cannabis is tetrahydrocannabinol (THC), which is used for treating a wide range of medical conditions, including glaucoma, AIDS wasting, neuropathic pain, treatment of spasticity associated with multiple sclerosis, fibromyalgia and chemotherapy-induced nausea. THC is also effective in the treatment of allergies, inflammation, infection, epilepsy, depression, migraine, bipolar disorders, anxiety disorder, drug dependency and drug withdrawal syndromes.

Additional pharmacologically-active cannabinoids include cannabidiol (CBD), an isomer of THC, which is a potent antioxidant and anti-inflammatory compound known to provide protection against acute and chronic neurodegeneration; cannabigerol (CBG), found in high concentrations in hemp, which acts as a high affinity a2-adrenergic receptor agonist, moderate affinity 5-HT_{1A} receptor antagonist and low affinity CB1 receptor antagonist, and possibly has anti-depressant activity; and cannabichromene (CBC), which possesses anti-inflammatory, anti-fungal and anti-viral properties. Many cannabinoids have therapeutic potential in a variety of diseases and may play a relevant role in pharmacology.

The primary method used to deliver marijuana into a patient's system is by smoking the marijuana. However, smoking increases an individual's risk for cancer, lung damage and emphysema. Furthermore, since marijuana does contain high levels of the psychoactive drug Δ⁹-THC, there has been considerable debate whether the potential health benefits of smoking marijuana are outweighed by the associated health risks.

Oral administration is the easiest and most convenient route for non-invasive drug administration. However, cannabinoids are highly lipophilic, meaning that they are soluble in lipids and some organic solvents while being substantially insoluble or only sparsely soluble in water. Cannabinoids are soluble in highly non-polar solvents (i.e., in substances such as chloroform, dichloromethane and high concentrations of alcohol); they also have limited solubility in glycols. Some of these solvents are pharmaceutically unacceptable, and the pharmaceutically acceptable solvents need to be used in high concentrations to produce solutions. Moreover, solubility in some of these solvents imposes a ceiling on the dose that can be given using conventional pharmaceutical methods of formulation. As such, the poor water-solubility of cannabinoids results in major difficulties in formulation and presents a major challenge to consistent drug delivery.

Furthermore, when administered orally in the form of an oil solution or some kind of water and/or oil suspension or emulsion, lipophilic compounds usually show poor bioavailability. For example, Δ⁹-THC is almost completely absorbed (90% to 95%) after a single oral dose. However, due to the combined effect of first pass, hepatic metabolism, and high lipid solubility, only about 10% to 20% of an administered dose reaches systemic circulation.

Another impediment to the medicinal use of cannabis is its well-known psychotropic side effects. Recent developments suggest that several cannabinoids exert very weak or no psychotropic effects. These include cannabidiol (CBD), cannabigerol (CBG), cannabichromene (CBC), Δ⁹-tetrahydrocannabivarin (Δ⁹-THCV), cannabidivarin (CBDV) as well as cannabinoid acids such as Δ⁹-tetrahydrocannabinolic acid (Δ⁹-THCA) and cannabidiolic acid (CBDA). These non-psychotropic cannabinoids have been shown to exert a wide range of pharmacological effects and could potentially be used to produce formulations with reduced or no psychotropic side effects.

Accordingly, there is a need for developing oral formulations of cannabinoids with enhanced bioavailability. Given the numerous cannabinoids with pharmacological activities, there is also a need to develop formulations with different ratios of two or more cannabinoids. Additionally, there is a need for developing cannabinoid formulations with reduced or no psychotropic side effects.

### SUMMARY OF THE INVENTION

Implementations described and claimed herein address the foregoing bioavailability problems by providing cannabinoid self-emulsifying drug delivery systems (SEDDS) having at least one cannabinoid; at least one lipophilic carrier with surfactant and solubilizing properties; at least one oil-soluble antioxidant; at least one water-soluble antioxidant; and a carrier. Implementations described and claimed herein also address the foregoing problems of THC's psychotropic side effects by providing cannabinoid SEDDS that include at least one cannabinoid exerting very weak or no psychotropic effects; at least one lipophilic carrier with surfactant and solubilizing properties; at least one oil-soluble antioxidant; at least one water-soluble antioxidant; and a carrier. These cannabinoid SEDDS oral formulations have enhanced bioavailability when compared with the same cannabinoids administered orally in the form of an oil solution or a water/oil suspension or emulsion.

In one embodiment, the SEDDS has one or more cannabinoid selected from tetrahydrocannabinol (THC), cannabidiol (CBD), cannabigerol (CBG), cannabichromene (CBC), Δ9-tetrahydrocannabivarin (Δ⁹-THCV), cannabidivarin (CBDV), Δ⁹-tetrahydrocannabinolic acid (Δ⁹-THCA), and cannabidiolic acid (CBDA); at least one lipophilic carrier selected from lauroyl polyoxyl-32 glycerides, caprylic/capric triglycerides, caprylic/capric/diglyceryl succinate, arachis oil, castor oil, cetosteryl alcohol, corn oil, cottonseed oil, glyceryl behenate, glycerol, maize propylene glycol monolaurate, olive oil, palm oil, propylene glycol diester of caprylic/capric acid, sesame oil, soybean oil, stearic acid, or steryl alcohol; at least one oil-soluble antioxidant selected from alpha tocopherol acetate, ascorbyl palmitate, butylated hydroxyanisole, butylated hydroxytoluene, or carotene; at least one water-soluble antioxidant selected from Vitamin E TPGS or polysorbate 80; and a gelatin or hypromellose capsule shell as a carrier.

In one embodiment, the SEDDS contains THC; lauroyl polyoxyl-32 glycerides and caprylic/capric triglycerides; alpha tocopherol acetate and ascorbyl palmitate; Vitamin E TPGS; and a gelatin or hypromellose capsule shell.

Such embodiments may comprise about 1.7 mg/dose to about 9.5 mg/dose THC, about 73% wt/wt to about 87.63% wt/wt lauroyl polyoxyl-32 glycerides, about 10% wt/wt caprylic/capric triglycerides, about 0.05 wt/wt to about 5.00 wt/wt alpha tocopherol acetate, about 0.10% wt/wt ascorbyl palmitate, and about 0.05% wt/wt to about 5.00% wt/wt Vitamin E TPGS.

In an alternative embodiment, the SEDDS contains THC and CBD; lauroyl polyoxyl-32 glycerides and caprylic/capric triglycerides; alpha tocopherol acetate and ascorbyl palmitate; Vitamin E TPGS; and a gelatin or hypromellose capsule shell.

Such alternative embodiments may comprise about 1.7 mg/dose to about 9.5 mg/dose THC, about 5.0 mg/dose to about 50.0 mg/dose CDB, about 73% wt/wt to about 87.63% wt/wt lauroyl polyoxyl-32 glycerides, about 10% wt/wt caprylic/capric triglycerides, about 0.05% wt/wt to about 5.00% wt/wt alpha tocopherol acetate, about 0.10% wt/wt ascorbyl palmitate, and about 0.05% wt/wt to about 5.00% wt/wt Vitamin E TPGS.

In one embodiment, the SEDDS formulation contains: about 3.3 mg/dose THC, about 50.0 mg/dose CBD, about 73.00% wt/wt lauroyl polyoxyl-32 glycerides, about 10.00% wt/wt caprylic/capric triglycerides, about 5.00 wt/wt alpha tocopherol acetate, about 0.10% wt/wt ascorbyl palmitate, and about 0.05% wt/wt Vitamin E TPGS.

In one embodiment, the cannabinoid SEDDS formulation contains: about 1.7 mg/dose THC, about 25.0 mg/dose CBD, about 73.00% wt/wt lauroylpolyoxyl-32 glycerides, about 10.00% wt/wt caprylic/capric triglycerides, about 5.00 wt/wt alpha tocopherol acetate, about 0.10% wt/wt ascorbyl palmitate, and about 0.05% wt/wt Vitamin E TPGS.

In one embodiment, the cannabinoid SEDDS formulation contains: about 9.5 mg/dose THC, about 9.5 mg/dose CBD, about 80.52% wt/wt lauroyl polyoxyl-32 glycerides, about 10.00% wt/wt caprylic/capric triglycerides, about 5.00 wt/wt alpha tocopherol acetate, about 0.10% wt/wt ascorbyl palmitate, and about 0.05% wt/wt Vitamin E TPGS.

In one embodiment, the cannabinoid SEDDS formulation contains: about 5.0 mg/dose THC, about 5.0 mg/dose CBD, about 80.41% wt/wt lauroyl polyoxyl-32 glycerides, about 10.00% wt/wt caprylic/capric triglycerides, about 5.00 wt/wt alpha tocopherol acetate, about 0.10% wt/wt ascorbyl palmitate, and about 0.05% wt/wt Vitamin E TPGS.

In one embodiment, the cannabinoid SEDDS formulation contains: about 9.5 mg/dose THC, about 87.74% wt/wt lauroyl polyoxyl-32 glycerides, about 10.00% wt/wt caprylic/capric triglycerides, about 5.00% wt/wt alpha tocopherol acetate, about 0.10% wt/wt ascorbyl palmitate, and about 0.05% wt/wt Vitamin E TPGS.

In one embodiment, the cannabinoid SEDDS formulation contains: about 5.0 mg/dose THC, about 87.63% wt/wt lauroyl polyoxyl-32 glycerides, about 10.00% wt/wt caprylic/capric triglycerides, about 5.00% wt/wt alpha tocopherol acetate, about 0.10% wt/wt ascorbyl palmitate, and about 0.05% wt/wt Vitamin E TPGS.

In one embodiment, the SEDDS formulation contains: about 3.3 mg/dose THC, about 50.0 mg/dose CBD, about 73.00% wt/wt lauroyl polyoxyl-32 glycerides, about 10.00% wt/wt caprylic/capric triglycerides, about 0.05 wt/wt alpha tocopherol acetate, about 0.10% wt/wt ascorbyl palmitate, and about 5.00% wt/wt Vitamin E TPGS.

In one embodiment, the cannabinoid SEDDS formulation contains: about 1.7 mg/dose THC, about 25.0 mg/dose CBD, about 73.00% wt/wt lauroylpolyoxyl-32 glycerides, about 10.00% wt/wt caprylic/capric triglycerides, about 0.05 wt/wt alpha tocopherol acetate, about 0.10% wt/wt ascorbyl palmitate, and about 5.00% wt/wt Vitamin E TPGS.

In one embodiment, the cannabinoid SEDDS formulation contains: about 9.5 mg/dose THC, about 9.5 mg/dose CBD, about 80.52% wt/wt lauroyl polyoxyl-32 glycerides, about 10.00% wt/wt caprylic/capric triglycerides, about 0.05 wt/wt alpha tocopherol acetate, about 0.10% wt/wt ascorbyl palmitate, and about 5.00% wt/wt Vitamin E TPGS.

In one embodiment, the cannabinoid SEDDS formulation contains: about 5.0 mg/dose THC, about 5.0 mg/dose CBD, about 80.41% wt/wt lauroyl polyoxyl-32 glycerides, about 10.00% wt/wt caprylic/capric triglycerides, about 0.05 wt/wt alpha tocopherol acetate, about 0.10% wt/wt ascorbyl palmitate, and about 5.00% wt/wt Vitamin E TPGS.

In one embodiment, the cannabinoid SEDDS formulation contains: about 9.5 mg/dose THC, about 87.74% wt/wt lauroyl polyoxyl-32 glycerides, about 10.00% wt/wt caprylic/capric triglycerides, about 0.05% wt/wt alpha tocopherol acetate, about 0.10% wt/wt ascorbyl palmitate, and about 5.00% wt/wt Vitamin E TPGS.

In one embodiment, the cannabinoid SEDDS formulation contains: about 5.0 mg/dose THC, about 87.63% wt/wt lauroyl polyoxyl-32 glycerides, about 10.00% wt/wt caprylic/capric triglycerides, about 0.05% wt/wt alpha tocopherol acetate, about 0.10% wt/wt ascorbyl palmitate, and about 5.00% wt/wt Vitamin E TPGS.

The above-described cannabinoid SEDDS formulations allow for the oral administration of cannabinoids achieving sufficiently high oral bioavailability to treat or prevent a disease, condition, or symptom of a disease. The diseases or conditions that are prevented or treated include, but are not limited to, glaucoma, AIDS wasting, neuropathic pain, treatment of spasticity associated with multiple sclerosis, fibromyalgia and chemotherapy-induced nausea, allergies, inflammation, infection, epilepsy, depression, migraine, bipolar disorders, anxiety disorder, drug dependency and drug withdrawal syndromes.

Other implementations are also described and recited herein.

### DESCRIPTION OF THE DRAWINGS

FIG. 1 presents concentrations of THC in blood drawn from beagle dogs dosed with a commercial tablet (squares) or a SEDDS formulation (circles), each containing about 2.5 mg of cannabinoids. Blood samples were drawn at the times indicated after dosing (Hours) and THC concentration measured by LC/MS/MS assay (ng/ml THC). Each data point represents the mean average of four subject animals.

### DETAILED DESCRIPTION OF THE INVENTION

It is to be appreciated that certain aspects, modes, embodiments, variations and features of the invention are described below in various levels of detail in order to provide a substantial understanding of the present invention.

### Definitions

The definitions of certain terms as used in this specification are provided below. Unless defined otherwise, all technical and scientific terms used herein generally have the same meaning as commonly understood by one of ordinary skill in the art to which this invention belongs.

As used in this specification and the appended claims, the singular forms “a,” “an” and “the” include plural referents unless the content clearly dictates otherwise. For example, reference to “a cell” includes a combination of two or more cells, and the like.

As used herein, the term “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

As used herein, the term “subject” refers to a mammal, including but not limited to a dog, cat, horse, cow, pig, sheep, goat, rodent, or primate. Subjects can be house pets (e.g., dogs, cats), agricultural stock animals (e.g., cows, horses, pigs, etc.), laboratory animals (e.g., mice, rats, rabbits, etc.), but are not so limited. Subjects include human subjects. The human subject may be a pediatric, adult, or a geriatric subject. The human subject may be of either sex.

As used herein, the terms “effective amount” and “therapeutically-effective amount” include an amount sufficient to prevent or ameliorate a manifestation of disease or medical condition, such as glaucoma, AIDS wasting, neuropathic pain, treatment of spasticity associated with multiple sclerosis, fibromyalgia and chemotherapy-induced nausea, allergies, inflammation, infection, epilepsy, depression, migraine, bipolar disorders, anxiety disorder, drug dependency and drug withdrawal syndromes. It will be appreciated that there will be many ways known in the art to determine the effective amount for a given application. For example, the pharmacological methods for dosage determination may be used in the therapeutic context. In the context of therapeutic or prophylactic applications, the amount of a composition administered to the subject will depend on the type and severity of the disease and on the characteristics of the individual, such as general health, age, sex, body weight and tolerance to drugs. It will also depend on the degree, severity and type of disease. The skilled artisan will be able to determine appropriate dosages depending on these and other factors. The compositions can also be administered in combination with one or more additional therapeutic compounds.

### Self-Emulsifying Drug Delivery System

Self-Emulsifying Drug Delivery System (SEDDS) is a solid or liquid dosage form comprising an oil phase, a surfactant and a co-surfactant, characterized primarily in that said dosage form can form oil-in-water emulsion spontaneously in the gastrointestinal tract or at ambient temperature (referring generally to body temperature, namely 37° C.) with mild stirring. When a SEDDS enters the gastrointestinal tract, it is initially self-emulsified as emulsion droplets and rapidly dispersed throughout the gastrointestinal tract, and thus reducing the irritation caused by the direct contact of the drug with the mucous membrane of the gastrointestinal tract. In the gastrointestinal tract, the structure of the emulsion microparticulates will be changed or destroyed. The resulting microparticulates of micrometer or nanometer level can penetrate into the mucous membrane of the gastrointestinal tract, and the digested oil droplets enter the blood circulation, thereby significantly improving the bioavailability of the drug. The self-emulsifying drug delivery system is predominantly employed with respect to lipid-soluble and less water-soluble drugs, such as cannabinoids. It increases the stability and the bioavailability of the lipophilic drugs, provides a more consistent temporal profile of drug absorption, protects the drugs from the hostile environment in the gastro-intestinal tract, eliminates food effects, and allows for dose escalation, thereby improving efficacy and safety.

### Cannabinoids

Cannabinoids are a group of extracellular signaling molecules. Signals from these molecules are mediated in animals by two G-protein coupled receptors, Cannabinoid Receptor 1 (CB₁) and Cannabinoid Receptor 2 (CB₂). CB₁ is expressed most abundantly in the neurons of the central nervous system (CNS) but is also present at lower concentrations in a variety of peripheral tissues and cells (Matsuda, et al. (1990) Nature 346:561-564). In contrast, CB₂ is expressed predominantly, although not exclusively, in non-neural tissues, e.g., in hematopoietic cells, endothelial cells, osteoblasts, osteoclasts, the endocrine pancreas, and cancerous cell lines (Munro, et al. (1993) Nature 365:61-65; and as reviewed in Pacher, et al. (2006) Pharmacol. Rev. 58(3): 389-462). As such, CB₁ is believed to be primarily responsible for mediating the psychotropic effects of cannabinoids on the body, whereas CB₂ is believed to be primarily responsible for most of their non-neural effects.

The well-known psychotropic effects of Δ⁹-THC have greatly limited its clinical use. However, as described above, the plant Cannabis contains many cannabinoids with weak or no psychoactivity that, therapeutically, might be more promising than Δ⁹-THC, or can be combined with lower doses of Δ⁹-THC to produce equivalent therapeutic benefits. The cannabinoid SEDDS of the present invention are helpful in addressing the adverse psychotropic effects of Δ⁹-THC.

### Neuropathic Pain

Neuropathic pain is a complex, chronic pain state that usually is accompanied by tissue injury. With neuropathic pain, the nerve fibers themselves may be damaged, dysfunctional, or injured. These damaged nerve fibers send incorrect signals to other pain centers. The impact of nerve fiber injury includes a change in nerve function both at the site of injury and areas around the injury.

Neuropathic pain often seems to have no obvious cause; but, some common causes of neuropathic pain include: alcoholism; amputation; back, leg, and hip problems; chemotherapy; diabetes; facial nerve problems; HIV infection or AIDS; multiple sclerosis; shingles; and spine surgery. Neuropathic pain symptoms may include: shooting and burning pain or tingling and numbness.

Standard pain treatments include the use of opioids, antidepressants, botulinum toxin type A, dietary supplements, and neuromodulation with deep-brain stimulation, motor cortex stimulation, and spinal cord stimulators and intrathecal pumps for the local delivery of opioids. Unfortunately, neuropathic pain often responds poorly to standard pain treatments and occasionally may get worse instead of better over time. Neuropathic pain can be very difficult to treat with only 40-60% of people achieving partial relief (Dworkin, et al. (2007) “Pharmacologic management of neuropathic pain: evidence-based recommendations.” Pain 132 (3): 237-51). For some people, it can lead to serious disability.

Cannabis and a number of cannabinoid receptor agonists appear to be effective for neuropathic pain (see, e.g., Grotenhermen and Müller-Vahl (July 2012) “The therapeutic potential of cannabis and cannabinoids.” Deutsches Arzteblatt international 109 (29-30): 495-501; Leung (July-August 2011). “Cannabis and its derivatives: review of medical use.” Journal of the American Board of Family Medicine 24 (4): 452-62).

Adverse effects of cannabinoids that have limited their use in the long-term control of neuropathic pain management include CNS depression, cardiovascular effects, and especially psychoactive side effects (Campbell, et al. (2001) “Are cannabinoids an effective and safe treatment option in the management of pain? A qualitative systematic review” BMJ 323 (7303): 13-6). Potential weight gain and possible harmful psychological effects are also of concern with long term use of cannabinoids (Vickers and Kennett (March 2005) “Cannabinoids and the regulation of ingestive behavior.” Curr Drug Targets 6 (2): 215-23). These adverse effects are addressed by the cannabinoid SEDDS of the present invention.

### EXAMPLES

### Example 1

### General Methods for Preparing a Cannabinoid Self-Emulsifying Drug Delivery System

The cannabinoid SEDDS are prepared by pre-melting Lauroyl polyoxyl-32 glycerides and Vitamin E TPGS at 40° C. to 70° C. Once melted, Lauroyl polyoxyl-32 glycerides and Vitamin E TPGS are added into a jacketed mixer set at 60° C. Caprylic/capric triglyceride, Alpha tocopherol acetate, and ascorbyl palmitate are then added to the jacketed mixer with constant agitation. Mixing is continued until the ascorbyl palmitate is completely dissolved.

Once the ascorbyl palmitate is completely dissolved, the cannabis extract/distillate is added to the jacketed mixer and mixed until a homogeneous solution is obtained. Representative cannabinoid SEDDS formulations A-F are described in Table 1, which summarizes the composition of each formulation tested.

Once the cannabinoid formulations are completely dissolved, each formulation in Table 1 is encapsulated in a Gelatin or HPMC capsule shell #1 or #0 (Capsugel, Morristown, N.J.) to produce the SEDDS.

TABLE 1

| | | Formulations | | | | | |
|---|---|---|---|---|---|---|---|
| Composition¹ | Amount | A | B | C | D | E | F |
| THC | mg/dose | 3.3 | 1.7 | 9.5 | 5.0 | 9.5 | 5.0 |
| CBD | mg/dose | 50.0 | 25.0 | 9.5 | 5.0 | 0.0 | 0.0 |
| Gelucire 44/14 | % wt/wt | 73.00 | 73.00 | 80.52 | 80.41 | 82.74 | 82.63 |
| Captex 300 | % wt/wt | 10.00 | 10.00 | 10.00 | 10.00 | 10.00 | 10.00 |
| Alpha tocopherol | % wt/wt | 5.00 | 5.00 | 5.00 | 5.00 | 5.00 | 5.00 |
| acetate | | | | | | | |
| TPGS 1000 | % wt/wt | 0.05 | 0.05 | 0.05 | 0.05 | 0.05 | 0.05 |
| Ascorbyl palmitate | % wt/wt | 0.10 | 0.10 | 0.10 | 0.10 | 0.10 | 0.10 |

¹The abbreviation and trade names used herein denote the following: THC refers to tetrahydrocannabinol; CBD refers to cannabidiol; Gelucire 44/14 refers to Lauroyl polyoxyl-32 glycerides (Gattefossé USA, Paramus, New Jersey); Captex 300 refers to Caprylic/Capric Triglyceride (Abitec, Columbus, Ohio); Alpha tocopherol acetate (Spectrum Chemical Mfg. Corp., New Brunswick, New Jersey or BASF Corp., Wyandotte, Michigan); TPGS 1000 refers to Vitamin E TPGS (PMC IsoChem, Vert le Petit, France); and Ascorbyl palmitate (Spectrum Chemical Mfg. Corp. or EMD Sigma, St. Louis, Missouri).

### Example 2

### Determination of Oral Bioavailability I

Subjects are selected for the in vivo oral bioavailability study. Subjects are fasted overnight prior to dosing. One of the SEDDS formulation (A-F) is orally administered to a first group of subjects (n=10). The same dose of cannabinoids is administered orally to second group of subjects (n=10) in the form of an oil solution. The same dose of cannabinoids is administered intravenously to third group of subjects (n=10).

Serial blood samples of 2 mL are obtained from subjects at 20 and 40 minutes and 1, 2, 4, 6, 8, 12, and 24 hours after dosing. These blood samples are analyzed using an HPLC or LC/MS/MS assay specific for the cannabinoids administered to each subject.

Samples were typically prepared by adding 25 μL aliquots of plasma to 200 μL of a solution of 0.1% formic acid in acetonitrile: methanol 1:1 containing THC-D₃ and 11-hydroxy THC-D₃ and extracted through a Biotage Isolute PLD+ plate (50 mg) extraction plate (Biotage LLC, Charlotte, N.C.). Extracted samples were analyzed by reverse phase liquid chromatography/tandem mass spectrometry (LC/MS/MS) in selective reaction monitoring (SRM) mode under optimized positive ion conditions for the detection of THC, 11-hydroxy THC and the deuterated internal standards. Analytes were separated by reverse phase HPLC employing a Waters BEH C8 (2.1×30 mm) column (Waters Corp., Milford, Mass.), under gradient conditions. The gradient begins with 50% MPA (10 mM ammonium acetate in water pH 4.8) for 0.5 minutes then increases to 95% MPB (0.1% acetic acid in methanol) in linear fashion over 1.5 minutes and then holds at 95% MPB for 1 minute before returning to the starting conditions. For MS/MS detection a Sciex API 5000 mass spectrometer (AB Sciex LLC, Redwood City, Calif.) was used to monitor four transition ions (THC 315.1→193.0, THC-D₃ 318.1→196.0, 11-hydroxy 331.1→193.0 and 11-hydroxy D₃ 334.1→196.0). The targeted quantitation range was 0.1 ng/mL to 100 ng/mL for THC and from 0.2 ng/mL to 200 ng/mL for 11-hydroxy THC.

Drug concentrations in the blood of the test subjects are plotted against the time after the drug is administered through an intravenous (iv) or oral route. The area under the plasma concentration-time curve (the AUCs) are recorded and integrated using the trapezoidal rule to calculate the absolute bioavailability according to the following formulae:

$$Absolute bioavailability ( \% ) = \frac{( AUC ) oral \text{/} Dose oral}{( AUC ) iv \text{/} Dose iv}$$

The self-emulsifying drug delivery system containing one of the formulations A-F achieves an oral bioavailability of the cannabinoids significantly higher than the same dose of cannabinoids administered orally in the form of an oil solution.

### Example 3

### Determination of Oral Bioavailability II

The plasma pharmacokinetics of a cannabinoid SEDDS formulation and a commercially available THC tablet were measured in a study utilizing non-naïve male Beagle dogs (n=4 for each test compound). In these tests the SEDDS formulation comprised 2.5 mg/dose THC, 82.01% wt/wt lauroyl polyoxyl-32 glycerides, 10.00% wt/wt caprylic/capric triglycerides, 0.05% wt/wt alpha tocopherol acetate, 0.10% wt/wt ascorbyl palmitate, and 5.00% wt/wt Vitamin E TPGS. The commercially available tablet formulation also contained 2.5 mg THC as an active ingredient. A 3 ml blood sample was drawn from each subject dog prior to oral administration of a single dose of the test compound. Blood samples were also taken at 0.5, 1, 2, 4, 6, 8, and 12 hours post-administration. Plasma was isolated from each sample and each sample split into two equal aliquots and stored at −80° C. until further analysis. LC/MS/MS determination of the concentration of THC was conducted at Pyxant Labs (Colorado Springs, Colo.).

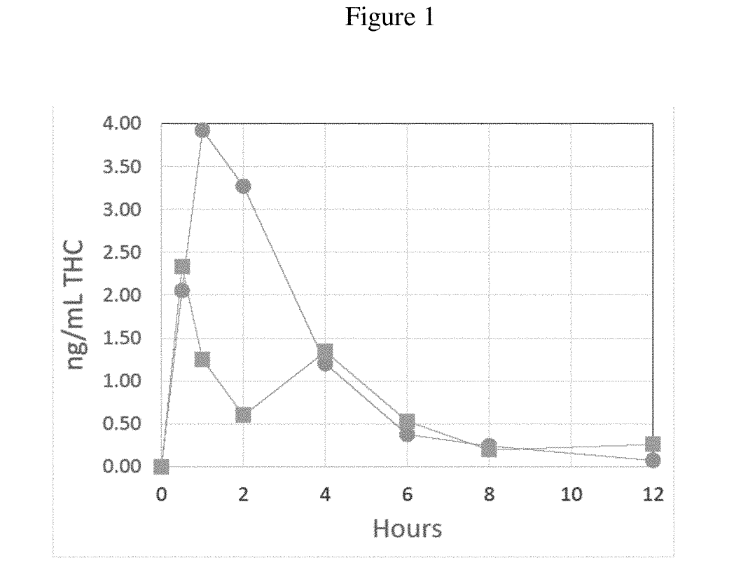

These data, presented in FIG. 1, indicate that the SEDDS formulation provides a markedly improved delivery relative to the commercial tablet in terms of rapidity and overall delivery efficiency of the active ingredient to the blood stream of the subject animals

### Example 4

### Assessment of Cannabinoid SEDDS in Management of Neuropathic Pain

As mentioned above, neuropathic pain often responds poorly to standard pain treatments, with only 40% to 60% of people achieving partial relief (Dworkin, et al. (2007) “Pharmacologic management of neuropathic pain: evidence-based recommendations.” Pain 132 (3): 237-51).

Subjects experiencing neuropathic pain and responding poorly to standard pain treatments are assessed for the effects of cannabinoid SEDDS on the management of their neuropathic pain (n=30). Subjects in a first group (n=15) are asked rate their pain level according to the pain scale in Table 2 prior to taking one of the cannabinoid SEDDS formulation A-F and 1, 4, 6, and 12 hours thereafter for seven consecutive days. The first group subjects are then asked to repeat the protocol but with the same dose of cannabinoids administered orally in the form of an oil solution. A second group of subjects (n=15) undergoes the same protocol but starting with the dose of cannabinoids administered orally in the form of an oil solution for seven consecutive days, followed by the cannabinoid SEDDS formulation.

Pain level scores over time are compared within subjects with respect to the two different forms of oral cannabinoid administration. The self-emulsifying drug delivery systems containing one of the formulations A-F achieves significantly lower pain level scores than the same dose of cannabinoids administered orally in the form of an oil solution.

TABLE 2
THE PAIN SCALE

0 - Pain free
Mild Pain - Nagging, annoying, but doesn't really interfere with daily
living activities
1 - Pain is very mild, barely noticeable. Most of the time you don't
think about it.
2 - Minor pain. Annoying and may have occasional stronger twinges.
3 - Pain is noticeable and distracting, however, you can get used to it
and adapt.
Moderate Pain - Interferes significantly with daily living activities
4 - Moderate pain. If you are deeply involved in an activity, it can be
ignored for a period of time, but is still distracting.
5 - Moderately strong pain. It can't be ignored for more than a few
minutes, but with effort you can still manage to work or participate in
some social activities.
6 - Moderately strong pain that interferes with normal daily activities.
Difficulty concentrating.
Severe Pain - Disabling; unable to perform daily living activities
7 - Severe pain that dominates your senses and significantly limits your
ability to perform normal daily activities or maintain social
relationships. Interferes with sleep.
8 - Intense pain. Physical activity is severely limited. Conversing
requires great effort.
9 - Excruciating pain. Unable to converse. Crying out and/or moaning
uncontrollably.
10 - Unspeakable pain. Bedridden and possibly delirious. Very few people
will ever experience this level of pain.

The foregoing written specification is considered to be sufficient to enable one skilled in the art to practice the present aspects and embodiments. The present aspects and embodiments are not to be limited in scope by examples provided, since the examples are intended as a single illustration of one aspect and other functionally equivalent embodiments are within the scope of the disclosure. Various modifications in addition to those shown and described herein will become apparent to those skilled in the art from the foregoing description and fall within the scope of the appended claims. The advantages and objects described herein are not necessarily encompassed by each embodiment. Those skilled in the art will recognize, or be able to ascertain using no more than routine experimentation, many equivalents to the specific embodiments described herein. Such equivalents are intended to be encompassed by the following claims.

All references disclosed herein are incorporated by reference in their entirety.

What is claimed is:

1. A cannabinoid self-emulsifying drug delivery system (SEDDS) comprising:
(i) at least one cannabinoid;
(ii) at least one lipophilic carrier with surfactant and solubilizing properties;
(iii) at least one oil-soluble antioxidant;
(iv) at least one water-soluble antioxidant; and
(v) a carrier.

2. The SEDDS of claim 1, wherein:
(i) the at least one cannabinoid is selected from the group consisting of: tetrahydrocannabinol (THC), cannabidiol (CBD), cannabigerol (CBG), cannabichromene (CBC), Δ⁹-tetrahydrocannabivarin (Δ⁹-THCV), cannabidivarin (CBDV), Δ⁹-tetrahydrocannabinolic acid (Δ⁹-THCA), and cannabidiolic acid (CBDA);
(ii) the at least one lipophilic carrier is selected from the group consisting of: lauroyl polyoxyl-32 glycerides, caprylic/capric triglycerides, caprylic/capric/diglyceryl succinate, arachis oil, castor oil, cetosteryl alcohol, corn oil, cottonseed oil, glyceryl behenate, glycerol, maize propylene glycol monolaurate, olive oil, palm oil, propylene glycol diester of caprylic/capric acid, sesame oil, soybean oil, stearic acid, and steryl alcohol;
(iii) the at least one oil-soluble antioxidant is selected from the group consisting of: alpha tocopherol acetate, ascorbyl palmitate, butylated hydroxyanisole, butylated hydroxytoluene, and carotene;
(iv) the at least one water-soluble antioxidant is selected from Vitamin E TPGS or polysorbate 80; and
(v) the carrier is selected from gelatin or hypromellose capsule shell.

3. The SEDDS of claim 1, wherein:
(i) the at least one cannabinoid is THC or CBD;
(ii) the at least one lipophilic carrier is lauroyl polyoxyl-32 glycerides and caprylic/capric triglycerides;
(iii) the at least one oil-soluble antioxidant is alpha tocopherol acetate and ascorbyl palmitate;
(iv) the at least one water-soluble antioxidant is Vitamin E TPGS; and
(v) the carrier is selected from gelatin or hypromellose capsule shell.

4. The cannabinoid SEDDS formulation of claim 3 comprising: about 1.7 mg/dose to about 9.5 mg/dose THC, about 73% wt/wt to about 87.63% wt/wt lauroyl polyoxyl-32 glycerides, about 10% wt/wt caprylic/capric triglycerides, about 0.05% wt/wt to about 5.00% wt/wt alpha tocopherol acetate, about 0.10% wt/wt ascorbyl palmitate, and about 0.05% wt/wt to about 5.00% wt/wt Vitamin E TPGS.

5. The cannabinoid SEDDS formulation of claim 3 comprising: about 1.7 mg/dose to about 9.5 mg/dose THC, about 5.0 mg/dose to about 50.0 mg/dose CDB, about 73% wt/wt to about 87.63% wt/wt lauroyl polyoxyl-32 glycerides, about 10% wt/wt caprylic/capric triglycerides, about 0.05 wt/wt to about 5.00 wt/wt alpha tocopherol acetate, about 0.10% wt/wt ascorbyl palmitate, and about 0.05% wt/wt to about 5.00% wt/wt Vitamin E TPGS.

6. The cannabinoid SEDDS formulation of claim 3 comprising: 3.3 mg/dose THC, 50.0 mg/dose CBD, 73.00% wt/wt lauroyl polyoxyl-32 glycerides, 10.00% wt/wt caprylic/capric triglycerides, 5.00 wt/wt alpha tocopherol acetate, 0.10% wt/wt ascorbyl palmitate, and 0.05 wt/wt Vitamin E TPGS.

7. The cannabinoid SEDDS formulation of claim 3 comprising: 1.7 mg/dose THC, 25.0 mg/dose CBD, 73.00% wt/wt lauroyl polyoxyl-32 glycerides, 10.00% wt/wt caprylic/capric triglycerides, 5.00 wt/wt alpha tocopherol acetate, 0.10% wt/wt ascorbyl palmitate, and 0.05 wt/wt Vitamin E TPGS.

8. The cannabinoid SEDDS formulation of claim 3 comprising: 9.5 mg/dose THC, 9.5 mg/dose CBD, 80.52% wt/wt lauroyl polyoxyl-32 glycerides, 10.00% wt/wt caprylic/capric triglycerides, 5.00 wt/wt alpha tocopherol acetate, 0.10% wt/wt ascorbyl palmitate, and 0.05 wt/wt Vitamin E TPGS.

9. The cannabinoid SEDDS formulation of claim 3 comprising: 5.0 mg/dose THC, 5.0 mg/dose CBD, 80.41% wt/wt lauroylpolyoxyl-32 glycerides, 10.00% wt/wt caprylic/capric triglycerides, 5.00% wt/wt alpha tocopherol acetate, 0.10% wt/wt ascorbyl palmitate, and 0.05 wt/wt Vitamin E TPGS.

10. The cannabinoid SEDDS formulation of claim 3 comprising: 5.0 mg/dose THC, 87.63% wt/wt lauroyl polyoxyl-32 glycerides, 10.00% wt/wt caprylic/capric triglycerides, 5.00 wt/wt alpha tocopherol acetate, 0.10% wt/wt ascorbyl palmitate, and 0.05% wt/wt Vitamin E TPGS.

11. The cannabinoid SEDDS formulation of claim 3 comprising: 3.3 mg/dose THC, 50.0 mg/dose CBD, 73.00% wt/wt lauroyl polyoxyl-32 glycerides, 10.00% wt/wt caprylic/capric triglycerides, 0.05 wt/wt alpha tocopherol acetate, 0.10% wt/wt ascorbyl palmitate, and 5.00 wt/wt Vitamin E TPGS.

12. The cannabinoid SEDDS formulation of claim 3 comprising: 1.7 mg/dose THC, 25.0 mg/dose CBD, 73.00% wt/wt lauroyl polyoxyl-32 glycerides, 10.00% wt/wt caprylic/capric triglycerides, 0.05 wt/wt alpha tocopherol acetate, 0.10% wt/wt ascorbyl palmitate, and 5.00 wt/wt Vitamin E TPGS.

13. The cannabinoid SEDDS formulation of claim 3 comprising: 9.5 mg/dose THC, 9.5 mg/dose CBD, 80.52% wt/wt lauroyl polyoxyl-32 glycerides, 10.00% wt/wt caprylic/capric triglycerides, 0.05 wt/wt alpha tocopherol acetate, 0.10% wt/wt ascorbyl palmitate, and 5.00 wt/wt Vitamin E TPGS.

14. The cannabinoid SEDDS formulation of claim 3 comprising: 5.0 mg/dose THC, 5.0 mg/dose CBD, 80.41% wt/wt lauroylpolyoxyl-32 glycerides, 10.00% wt/wt caprylic/capric triglycerides, 0.05 wt/wt alpha tocopherol acetate, 0.10% wt/wt ascorbylpalmitate, and 5.00 wt/wt Vitamin E TPGS.

15. The cannabinoid SEDDS formulation of claim 3 comprising: 5.0 mg/dose THC, 87.63% wt/wt lauroyl polyoxyl-32 glycerides, 10.00% wt/wt caprylic/capric triglycerides, 0.05 wt/wt alpha tocopherol acetate, 0.10% wt/wt ascorbyl palmitate, and 5.00% wt/wt Vitamin E TPGS.

16. The cannabinoid SEDDS of any one of claims 1-3, wherein the cannabinoid administered orally in the SEDDS has enhanced bioavailability when compared with the same cannabinoid administered orally in the form of an oil solution.
